(19) United States
(12) Patent Application Publication
(10) Pub. No.: US 2011/0303760 A1
(43) Pub. Date: Dec. 15, 2011

(54) APPARATUS AND METHOD FOR CONTROLLABLY RELEASING A SUBSTANCE

(76) Inventors: Ashok Joshi, Salt Lake City, UT (US); Truman Wold, Salt Lake City, UT (US); David Erekson, Toole, UT (US); John Howard Gordon, Salt Lake City, UT (US)

(21) Appl. No.: 12/950,053

(22) Filed: Nov. 19, 2010

Related U.S. Application Data
(60) Provisional application No. 61/354,132, filed on Jun. 11, 2010.

(51) Int. Cl. A61L 9/04

(52) U.S. Cl. 239/44

(57) ABSTRACT

An apparatus for controllably releasing a substance is disclosed herein. In one embodiment, such an apparatus includes a chamber to store a non-gaseous fluid. An outlet communicates with a bottom of the chamber to dispense the non-gaseous fluid. An inlet communicates with a top of the chamber to enable gas to flow into the chamber. To regulate the flow of non-gaseous fluid through the outlet, a regulator element is coupled to the inlet to regulate the flow of gas into the chamber. In certain embodiments, to provide more consistent release rates, the chamber includes a short wide portion situated above a relatively long narrow portion. Such a configuration generates more consistent head pressure and thus more consistent release rates for the non-gaseous fluid. A corresponding method is also disclosed herein.

### RELATED APPLICATIONS

This application claims priority to U.S. Provisional Patent No. 61/354,132 filed on Jun. 11, 2010 and entitled CONTROLLED RELEASE OF SUBSTANCES.

### FIELD OF THE INVENTION

This invention relates to apparatus and methods for controlling the release of substances into the atmosphere or other environments.

### BACKGROUND

Different types of devices have been designed to release substances into the atmosphere or other environments over a period of time. Examples of such devices include various types of air fresheners designed to release fragrances or deodorizing agents into the atmosphere. Other analogous devices include those that emanate or release insect repellants, pesticides, disinfectants, antimicrobial agents, medicines, and other beneficial agents.

Within the field of air fresheners, a wide variety of different devices exist. For example, scented candles and devices using flames or other heat sources may be used to heat and vaporize a fragrance for release into the atmosphere. Incense burners may be used to burn aromatic biotic materials to release fragrant smoke. Wall plug-ins may utilize piezoelectricity to aerosolize a fragrance or use heat to vaporize it. Fragrance-impregnated gels are widely used to release fragrances into the atmosphere as the gels evaporate. Wick and reed diffusers soaked with fragrances may be used to disperse the fragrances by evaporation. Fragrance-impregnated materials such as floor wax, paper, plastics, and wood may release fragrances into the atmosphere by offgassing. Nebulization systems may convert liquid fragrances into vapors without the use of heat.

Many of the air freshener devices discussed above, however, suffer from various shortcomings. For example, some devices may be mechanically complex and thus may be too unreliable or expensive to produce for mass consumption. Other devices may have safety issues. For example, scented candles or incense burners may create fire hazards and thus may not be suitable for long periods of unattended use. Other devices may release their fragrances too quickly, thereby making them unsuitable for long term use. Yet other devices may suffer from inconsistent release rates. For example, the release rates of fragrance-impregnated gels or materials may diminish over time, making them less effective as time passes.

In view of the foregoing, what are needed are apparatus and methods that address many of the shortcomings of the prior art. In particular, apparatus and methods are needed to controllably release substances in a simple, reliable, and inexpensive manner. Further needed are apparatus and methods to controllably release substances over long periods of time and with consistent release rates.

### SUMMARY

The invention has been developed in response to the present state of the art and, in particular, in response to the problems and needs in the art that have not yet been fully solved by currently available apparatus and methods. Accordingly, the invention has been developed to provide apparatus and methods to controllably release substances. The features and advantages of the invention will become more fully apparent from the following description and appended claims, or may be learned by practice of the invention as set forth hereinafter.

Consistent with the foregoing, an apparatus for controllably releasing a substance is disclosed herein. In one embodiment, such an apparatus includes a chamber to store a non-gaseous fluid. An outlet communicates with a bottom of the chamber to dispense the non-gaseous fluid. An inlet communicates with a top of the chamber to enable gas to flow into the chamber. To regulate the flow of non-gaseous fluid through the outlet, a regulator element is coupled to the inlet to regulate the flow of gas into the chamber. Such a regulator element may include one or more of a tube, a helical path, a gas-permeable membrane, or a solid having a channel formed therein, among others. In certain embodiments, to provide more consistent release rates, the chamber includes a short wide portion situated above a relatively long narrow portion. Such a configuration generates more consistent head pressure and thus more consistent release rates for the non-gaseous fluid.

A corresponding method is also disclosed and claimed herein.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings in which:

FIG. 1 is a perspective, cross-sectional view of one embodiment of an apparatus for controllably releasing a substance;

FIG. 2 shows one technique for housing the tube illustrated in FIG. 1 in a more compact space;

FIG. 3 is a perspective, cross-sectional view of an alternative embodiment of an apparatus for controllably releasing a substance;

FIG. 4 is a perspective, cross-sectional view of yet another alternative embodiment of an apparatus for controllably releasing a substance;

FIG. 5 is a perspective, cross-sectional view of an apparatus fitted within a collection tray, and a wicking emanator in communication with the collection tray to disperse non-gaseous fluid into the atmosphere;

FIG. 6 shows an apparatus dispensing a non-gaseous fluid into an emanator such as a heater, nebulizer, or piezo electric disperser;

FIG. 7 is a plot showing the amount of fluid delivered from an apparatus using two different lengths of 0.0025″ ID tubing over a three day period;

FIG. 8 is a plot showing the amount of fluid delivered from an apparatus using a 15″ length of 0.0025″ ID tubing over a ten day period;

FIG. 9 is a plot showing the amount of fluid delivered from an apparatus using a 3″ length of 0.0025″ ID tubing over a ten day period and in a non-temperature-controlled environment; and

FIG. 10 is a perspective, cross-sectional view of an alternative embodiment of an apparatus having a short wide portion situated above a relatively long narrow portion to generate more consistent head pressure.

### DETAILED DESCRIPTION OF THE INVENTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention. The presently described embodiments will be best understood by reference to the drawings, wherein like parts are designated by like numerals throughout.

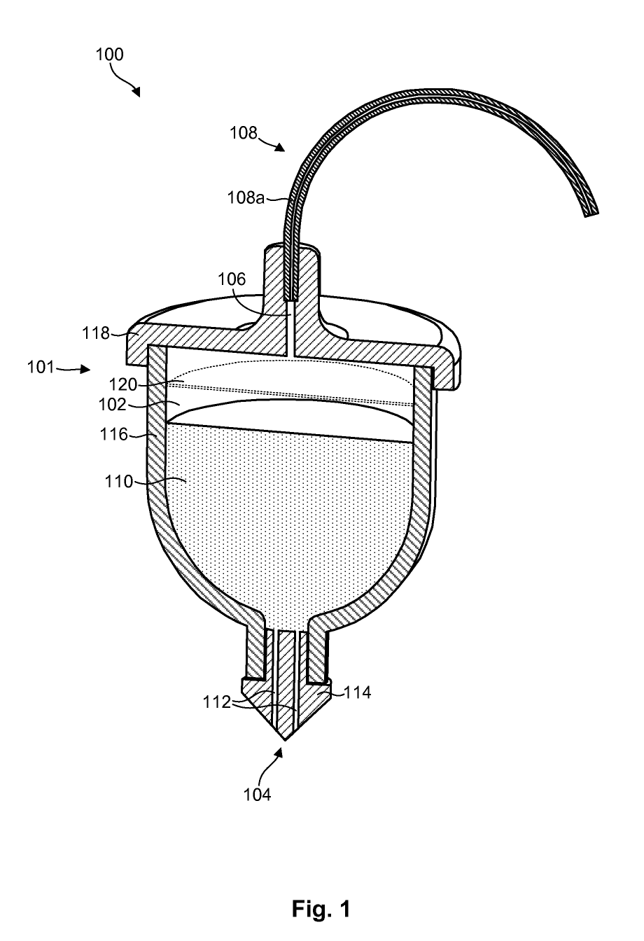

Referring to FIG. 1, a perspective, cross-sectional view of one embodiment of an apparatus 100 for controllably releasing a substance is illustrated. As shown, the apparatus 100 includes a housing 101, forming a chamber 102, to store a non-gaseous fluid 110 (e.g., a liquid). In selected embodiments, the non-gaseous fluid 110 contains a fragrance, a deodorizing agent, an insect repellant, a pesticide, a disinfectant, an antimicrobial agent, a medicine, and/or other beneficial agent. An outlet 104 communicates with a bottom of the chamber 102 to dispense the non-gaseous fluid 110. An inlet 106 communicates with a top of the chamber 102 to enable a gas (e.g., air) to flow into the chamber 102. A regulator element 108 is coupled to the inlet 106 to regulate the flow of gas through the inlet 106. This, in turn, will regulate the flow of non-gaseous fluid 110 through the outlet 104.

In operation, gravity may act on the non-gaseous fluid 110 to urge the non-gaseous fluid 110 through the outlet 104, which is located at or near the bottom of the chamber 102. As the non-gaseous fluid 110 is urged through the outlet 104, the pressure may be reduced within chamber 102. This reduced pressure may resist the pull of gravity and restrict the flow of non-gaseous fluid 110 through the outlet 104. The reduced pressure in the chamber 102 may also pull gas through the inlet 106 and regulator element 108 into the chamber 102. The regulator element 108 controls the rate that gas enters the chamber 102, thereby affecting the rate at which the non-gaseous fluid 110 is dispensed through the outlet 104.

In the illustrated embodiment, the regulator element 108 is a tube 108a coupled to the inlet 106. The length and inside diameter of the tube 108a may be varied to regulate the flow of gas into the chamber 102 and thus the flow of non-gaseous fluid 110 out of the chamber 102. Increasing the length of the tube 108a and/or decreasing the inside diameter of the tube 108a will decrease the rate that non-gaseous fluid 110 flows out of the chamber 102. Other factors, such as the viscosity of the non-gaseous fluid 110, may also be varied to adjust the flow rate of the non-gaseous fluid 110 out of the chamber 102. Increasing or decreasing the flow rate of the non-gaseous fluid 110 may be as simple as replacing the tube 108a with one that has different characteristics (e.g., length and/or diameter). In other embodiments, the tube 108a is non-replaceable, or the tube 108a and inlet 106 form a single integrated structure.

The outlet 104 may take on various shapes and forms. In the illustrated embodiment, the outlet 104 includes a pair of channels 112 integrated into a tip 114. A pair of channels 112 may be provided such that if one channel 112 becomes clogged or inoperable, the other channel 112 may continue to dispense the non-gaseous fluid 110. In certain embodiments, the tip 114 may be designed such that it can be removed, cleaned, and reattached, if needed. Alternatively, the tip 114 may be removed and replaced with a new tip 114 if it becomes clogged or inoperable. In certain embodiments, the outlet 104 may be placed at a location just above the bottom of the chamber 102. This may be useful in applications where sediments may tend to clog the outlet 104.

The housing 101 may also take on various shapes and forms. In the illustrated embodiment, the housing 101 is designed such that it includes a cup 116 and a cap 118. In selected embodiments, the cap 118 is attached permanently to the cup 116. In other embodiments, the cap 118 is removable to allow the chamber 102 to be refilled by removing and replacing the cap 118. This may allow the apparatus 100 to be reused more than once. In other embodiments, the apparatus 100 is simply thrown away when the non-gaseous fluid 110 in the chamber 102 is consumed.

In yet other embodiments, the cap 118 and cup 116 form a single integrated structure. In such embodiments, a port may be provided in the housing 101 to allow the chamber 102 to be filled with the non-gaseous fluid 110. The housing 101 (as well as other components of the apparatus 100) may be fabricated from any suitable materials having desired strength, flexibility, and/or cost. Various types of plastics may be suitable. Similarly, various fabrication techniques known in the art may be used to fabricate the apparatus 100.

In certain embodiments, a gas-permeable splash guard 120 is placed inside the chamber 102 to prevent the non-gaseous fluid 110 from splashing up and interfering with the inlet 106 or the regulator 108. Because the path through the regulator 108 and/or inlet 106 may be very narrow, the surface tension of any droplets on the path may be enough to block the flow of gas into the chamber 102. The splash guard 120 may ensure that the non-gaseous fluid 110 does not inadvertently make contact with and block the path. In selected embodiments, the splash guard 120 is made from a porous material such as a screen or porous membrane material.

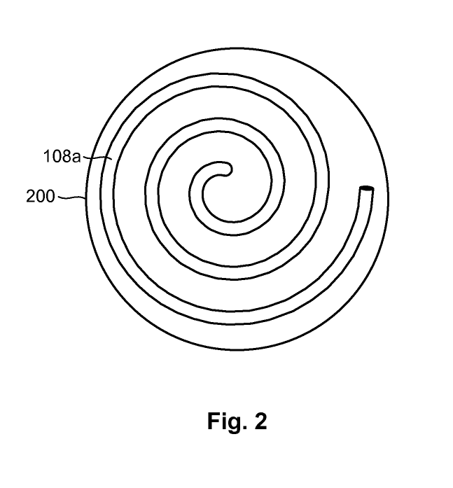

Referring to FIG. 2, in selected embodiments, the tube 108a illustrated in FIG. 1 may be coiled or formed in various tortuous shapes to make it more compact and fit into smaller spaces. For example, the tube 108a may be coiled and placed on the top of the cap 118. Alternatively, the tube 108a could be coiled and retained within a cavity or recess within the cap 118. In yet other embodiments, the tube 108a could be coiled and retained within the chamber 102. In this way, a long path length may be provided within a small space or area. FIG. 2 shows the tube 108a coiled to fit within a smaller circular shape 200. In certain embodiments, such a circular area 200 may be provided on top of, within, or beneath the cap 118.

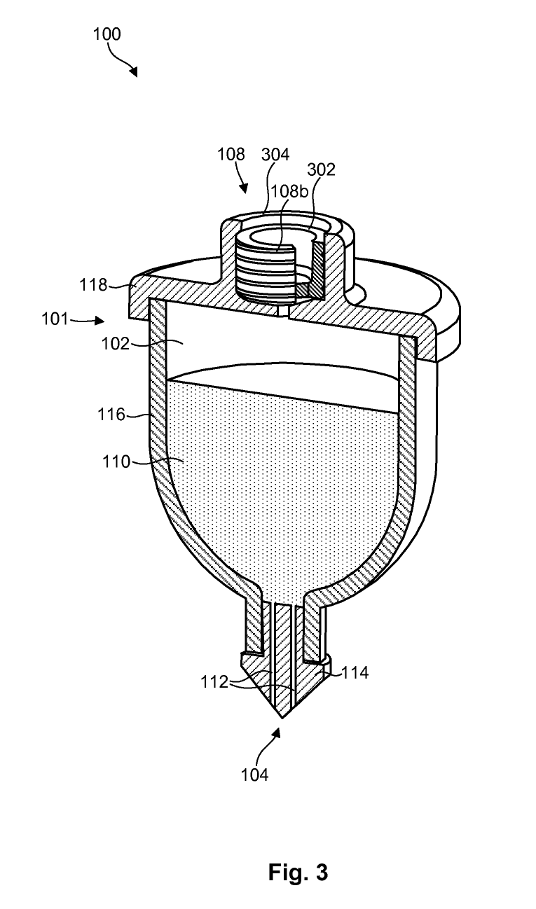

Referring to FIG. 3, a perspective, cross-sectional view of an alternative embodiment of an apparatus 100 for controllably releasing a substance is illustrated. In this embodiment, the regulator 108 includes a helical path 108b to regulate the flow of gas into the chamber 102. In the illustrated example, the helical path 108b is created by providing a cylindrical member 302 with a helical groove 108b formed around a circumference thereof. In certain embodiments, the helical groove 108b is formed on the cylindrical member 302 with a CNC lathe or other suitable device. Alternatively, the grooved cylindrical member 302 is formed using plastic injection molding. Once formed, the grooved cylindrical member 302 may be pressed or inserted into a larger cylindrical member 304 integrated into the housing 101 or cap 118. The grooved cylindrical member 302 may be retained within the larger cylindrical member 304 using a press fit, adhesive, or the like. The grooved cylindrical member 302 and larger cylindrical member 304 together form the helical path 108b. Like the coiled tube 108a previously discussed, the helical path 108b allows a long path to be contained within a more compact space.

The rate that the non-gaseous fluid 110 flows through the outlet 104 may be adjusted by changing the dimensions of the helical path 108b. The length of the helical path 108b may be adjusted by changing the number of rotations of the groove 108b around the inner cylindrical member 302. The depth and width of the groove 108b may also be modified to adjust the flow rate. In selected embodiments, a different inner cylindrical member 302 having different characteristics may be inserted into the outer cylindrical member 304 to change the rate that the non-gaseous fluid 110 flows through the outlet 104. In certain embodiments, the apparatus 100 may include multiple inner cylindrical members 302, each having different characteristics. A user may then select an inner cylindrical member 302 that provides a desired flow rate.

Other variations are also possible and within the scope of the invention. For example, in selected embodiments, several intertwined helical grooves 108b may be formed around the inner cylindrical member 302 to provide multiple paths to accommodate the flow of gas. This may provide redundancy if one path becomes clogged or otherwise inoperable. In other embodiments, several inner cylindrical members 302, each with a helical path 108b, may be placed on top of one another (in a series configuration) to regulate the flow of gas into the chamber 102. In yet other embodiments, several inner cylindrical members 302, each with a helical path 108b, may be arranged in parallel to regulate the flow of gas into the chamber 102.

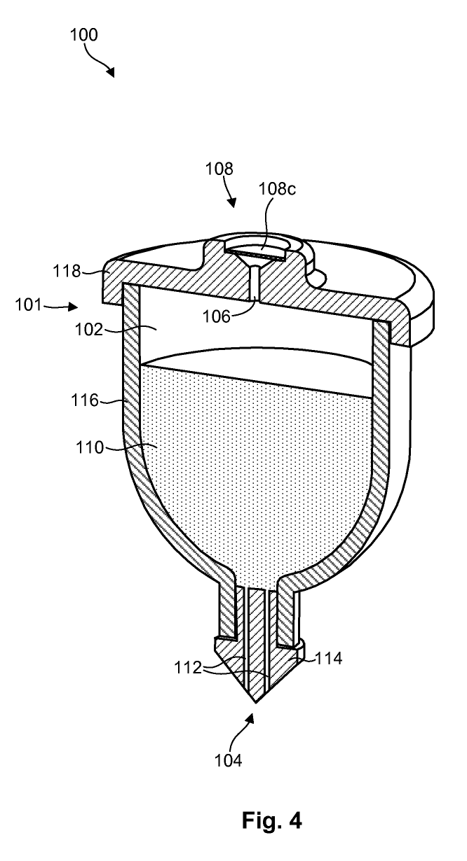

Referring to FIG. 4, a perspective, cross-sectional view of an alternative embodiment of an apparatus 100 for controllably releasing a substance is illustrated. In this embodiment, the regulator 108 includes a gas-permeable membrane 108c to regulate the flow of gas into the chamber 102. The gas-permeable membrane 108c may be porous or may be permeable to gases in the environment such as air. Examples of gas-permeable membranes 108c include membranes made from expanded films such as those made by Gortex. Other suitable materials for the membrane 108c include unsintered polytetrafloroethylene (PTFE), which is typically gas permeable, and elastomers such as polyurethane, which are also typically quite permeable. Other possible materials for the membrane 108c include porous polymers such as porous polyethylene or nylon.

The rate that gas flows into the chamber 102 may be adjusted by changing the characteristics of the gas-permeable membrane 108c. For example, the thickness, surface area, and porosity of the gas-permeable membrane 108c may be modified to adjust the flow rate. In selected embodiments, the gas flow rate into the chamber 102 may be adjusted by simply replacing the gas-permeable membrane 108c with another membrane 108c having desired characteristics. In certain embodiments, the gas-permeable membrane 108c is designed such that it is easily removed and replaced by a user. In certain embodiments, several gas-permeable membranes 108c may be arranged in a series or parallel configuration to provide a desired gas flow rate into the chamber 102, thereby providing a desired dispensing rate for the non-gaseous fluid 110.

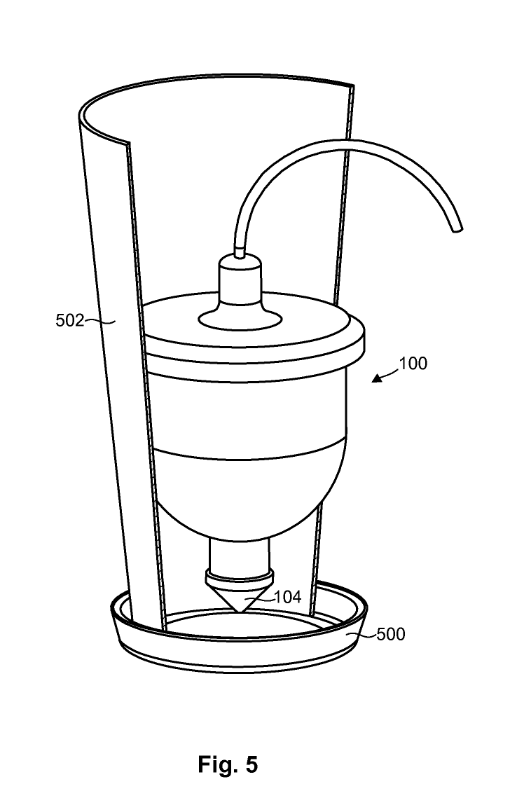

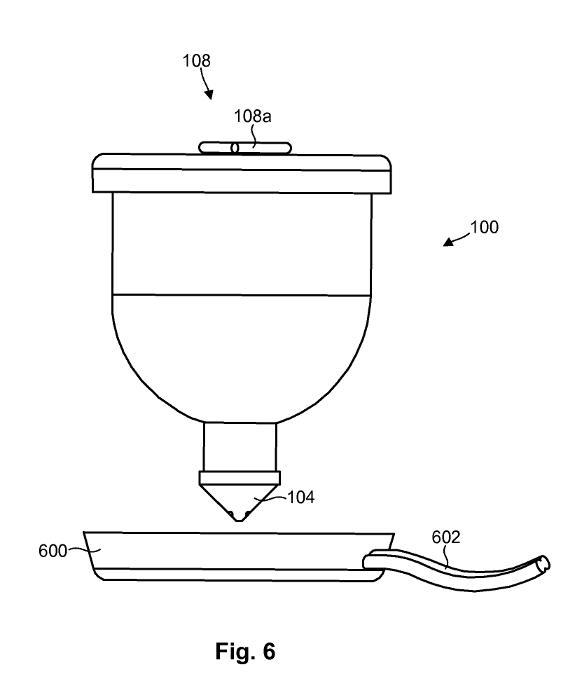

Referring to FIGS. 5 and 6, in selected embodiments, the apparatus 100 may be configured to release the non-gaseous fluid 110 into various types of emanators for dispersal into the surrounding atmosphere. Such emanators may include, for example, wicking devices, heaters, piezo devices, nebulizers, fans, or combinations thereof. FIG. 5 shows one embodiment of an apparatus 100 fitted within a liquid collection tray 500 and a wicking emanator 502 to evaporate the non-gaseous fluid 110 into the surrounding environment. More specifically, the non-gaseous fluid 110 flows through the outlet 104 of the apparatus 100 into the collection tray 500. A wicking emanator 502 contacts the liquid in the collection tray 500 and wicks to a large surface area such that the non-gaseous fluid 110 evaporates into the environment. The emanator 502 may be fabricated from wicking materials such as various kinds of cellulose or paper materials.

FIG. 6 shows one embodiment of an apparatus 100 communicating with an emanator 600 such as a heater, nebulizer, or piezo electric disperser. A power cord 602 may supply electrical power to the emanator 600. A heater may be used to heat the non-gaseous fluid 110 to promote evaporation. A nebulizer or piezo electric disperser, on the other hand, may convert the non-gaseous fluid 110 to a fine mist or spray to facilitate dispersal into the environment. In the illustrated embodiment, the regulator 108 is a coiled tube 108a located on top of the apparatus 100.

In selected embodiments, the emanator 600 is configured to disperse the non-gaseous fluid 110 at a rate faster than the dispersal rate of the apparatus 100. In some embodiments, for example, the emanator 600 may be configured to promptly disperse the non-gaseous fluid 110 as it is received from the apparatus 100. This will ensure that the non-gaseous fluid 110 does not build up in the emanator 600. This will also ensure that the regulator 108 is the primary means for controlling the rate at which the non-gaseous fluid 110 is dispersed into the atmosphere.

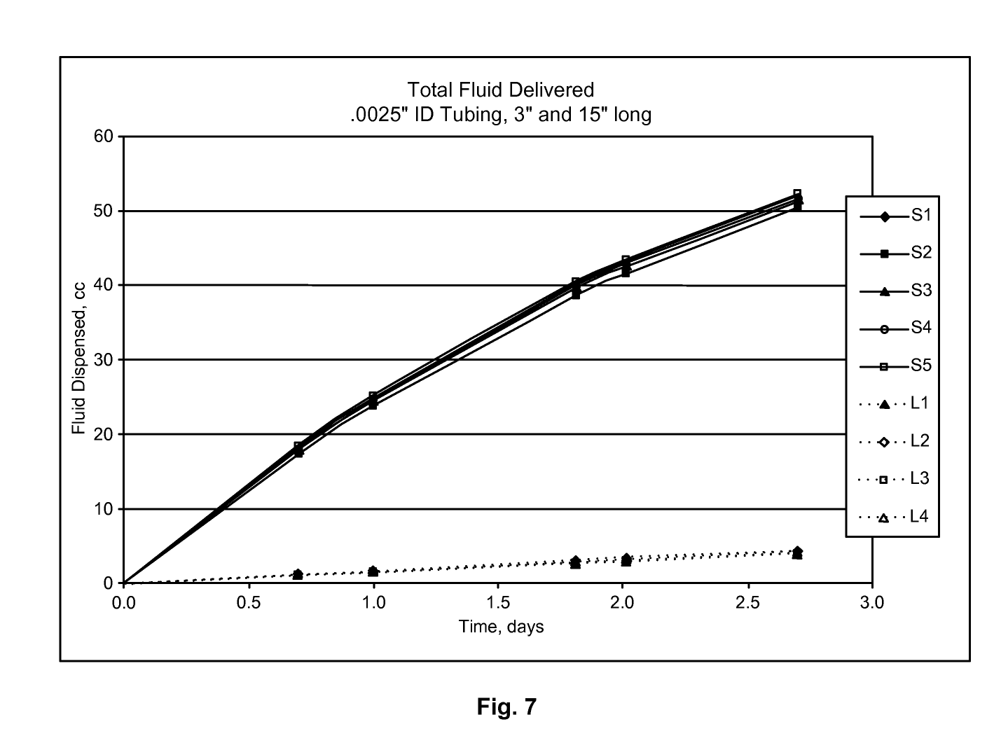

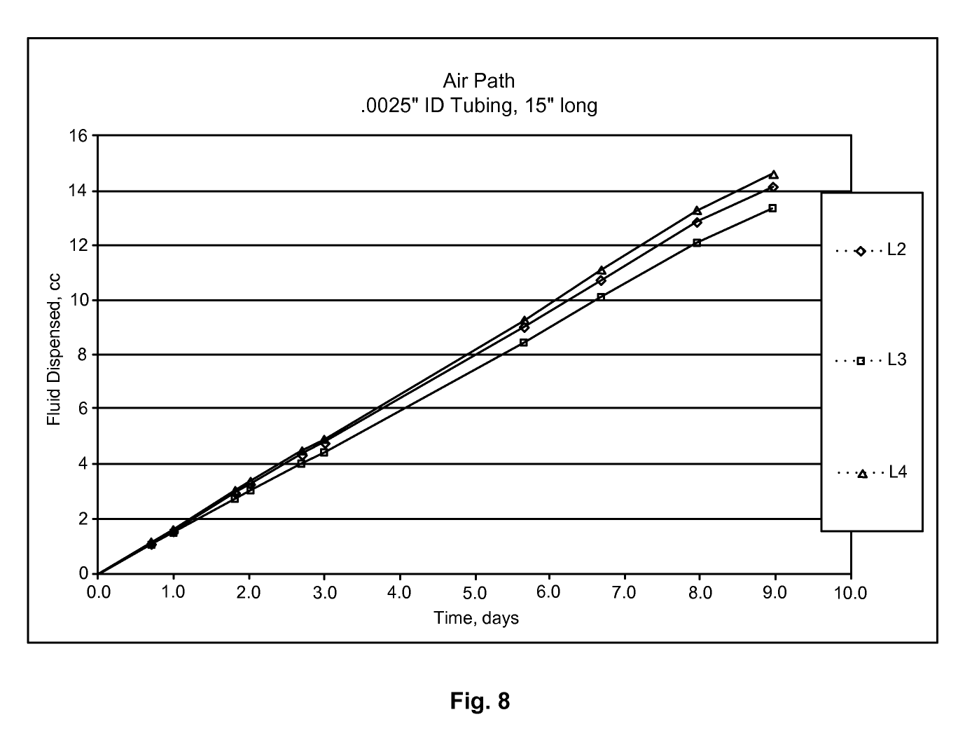

Referring to FIG. 7, a plot showing the amount of fluid delivered over time from an apparatus 100 similar to that illustrated in FIG. 1 is illustrated. The experiment was conducted using two different lengths of 0.0025″ ID tubing 108a over a three day period. The first length of tubing was 3″ and the second length of tubing was 15″. The experiment was repeated five times for the shorter 3″ length of tubing (runs S1-S5). The experiment was repeated four times for the longer 15″ length of tubing (runs L1-L4). The temperature did not fluctuate significantly during the experiments. Thus, pressure changes inside the chamber 102 as a result of changing temperatures were not significant. The non-gaseous fluid 110 was a fragrance commonly used in many public washrooms. The results of the experiment over a three day period are shown in FIG. 7. Extended results for the apparatus 100 using 15″ tubing over a ten day period are shown in FIG. 8.

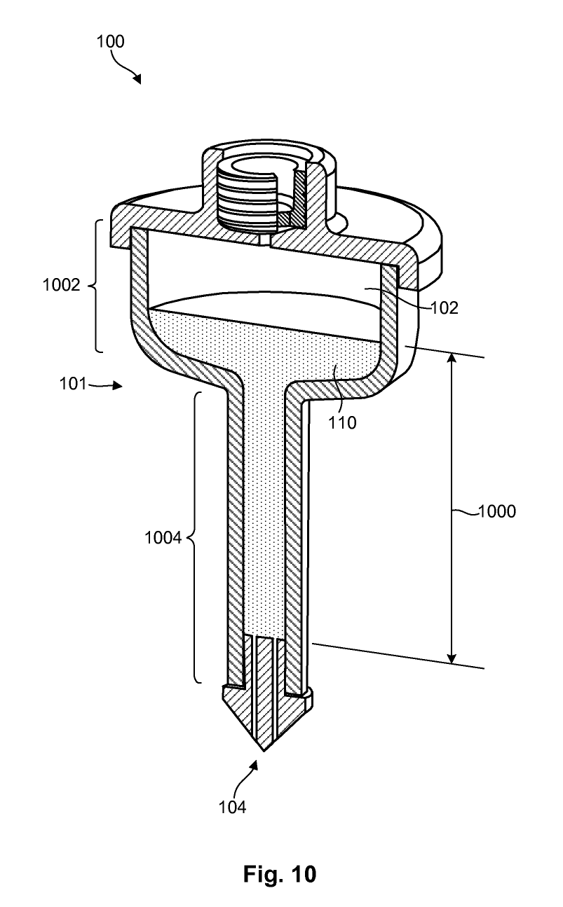

As can be observed from the plot shown in FIG. 7, the fluid dispensing rate of the apparatus 100 was very consistent for each length of tubing. The dispensing rate was also very linear, with only a slight upward bow in the data. The upward bow may be attributed to the fact that as the chamber 102 empties, the weight of non-gaseous fluid 110 in the chamber 102 is reduced. This will reduce the head pressure at the outlet 104, which will in turn reduce the dispensing rate of the non-gaseous fluid 110. One technique for mitigating this reduction in head pressure and increasing the consistency of the dispensing rate over time will be discussed in association with FIG. 10.

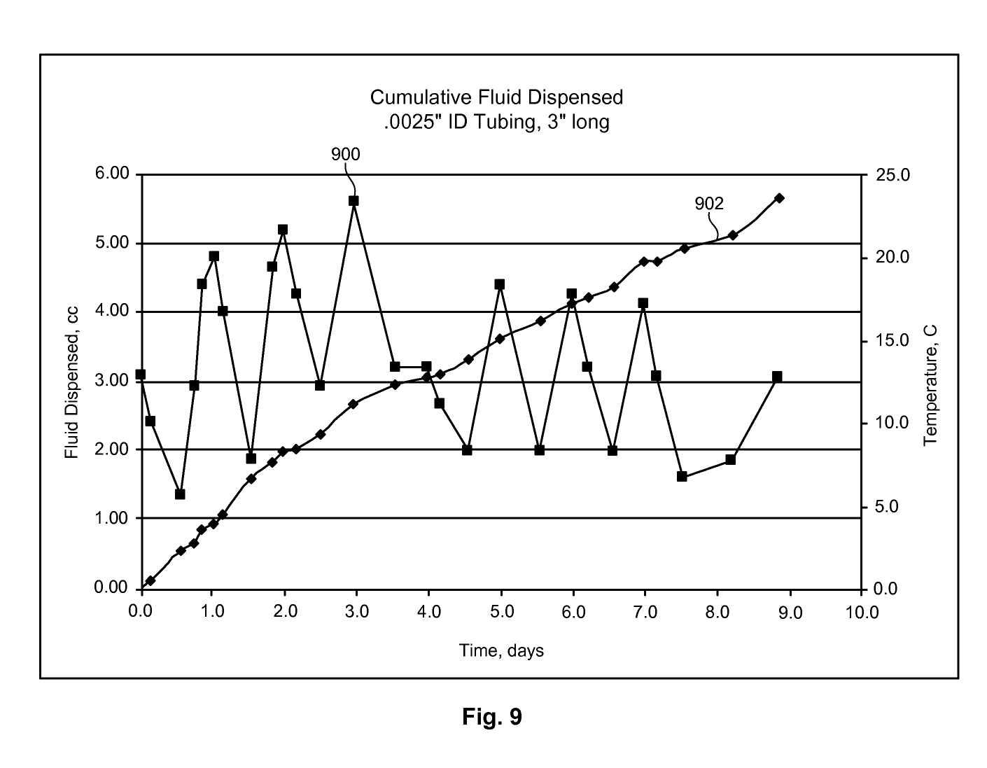

Referring to FIG. 9, a plot showing the amount of fluid delivered from an apparatus 100 using a 3″ length of 0.0025″ ID tubing over a ten day period is illustrated. During this experiment, the temperature of the apparatus 100 varied significantly by operating the apparatus 100 in a building with no temperature control. Such an operating environment may be similar to those encountered in cars, mobile restrooms, or in non-temperature-controlled buildings. The apparatus 100 and fragrance delivered were identical to the apparatus 100 and fragrance delivered in the experiments described in FIGS. 7 and 8, with the exception that the experiment exclusively used a 3″ length of 0.0025″ ID tubing. The plot line 900 shows the change in temperature over time and the plot line 902 shows the amount of fluid dispensed over time.

As can be observed from the plots 900, 902, the dispensing rate was fairly linear over the ten day measurement period even in the presence of significant temperature changes. This ability to maintain linearity may be attributed to the fact that the apparatus 100 is an open system. Thus, as pressure increases in the chamber 102 due to changing temperature, the pressure may be relieved through the inlet 106 and regulator 108 as opposed to forcing additional non-gaseous fluid 110 through the outlet 104. Thus, the open system may provide more consistent and reliable operation than a similar closed system. As mentioned above, this may provide significant advantages where the apparatus 100 is operated in environments with large temperature fluctuations, such as in cars, mobile restrooms, or non-temperature-controlled buildings.

Referring to FIG. 10, as mentioned above, as the chamber 102 empties, the head pressure at the outlet 104 is reduced, thereby reducing the dispensing rate of the non-gaseous fluid 110. FIG. 10 shows one embodiment of an apparatus 100 that may be used to mitigate the reduction in head pressure and increase the consistency of the dispensing rate as the chamber 102 empties. As illustrated, the height 1000 of the non-gaseous fluid 110 within the chamber 102 determines the head pressure at the outlet 104. Thus, in certain embodiments, the housing 101 may be designed such that the height 1000 varies as little as possible as the chamber 102 empties. This may be accomplished by designing the chamber 102 such that it includes a short wide portion 1002 and a relatively long narrow portion 1004. Due to its larger volume, most of the non-gaseous fluid 110 will be stored in the short wide portion 1002. As the chamber 102 empties, the height 1000 of the non-gaseous fluid 110 will decrease in the short wide portion 1002. However, due to the long narrow portion 1004, the percentage of the overall height 1000 will change very little until the height reaches the long narrow portion 1004. As long as the height 1000 of the non-gaseous fluid 110 remains in the short wide portion 1002, the head pressure will be maintained at a more consistent level as the chamber 102 empties. This, in turn, will ensure that the dispensing rate of the apparatus 100 is more consistent over time.

One of skill in the art will recognize that the apparatus 100 illustrated in FIG. 10 may be designed in many different shapes and forms without departing from the characteristics or principles of operation described herein. Thus, the shape and form of the apparatus 100 illustrated in FIG. 10 is provided only by way of example and not limitation. Other shapes and forms may achieve a similar result and thus are intended to be encompassed with the scope of the invention.

The present invention may be embodied in other specific forms without departing from its basic principles or essential characteristics. The described embodiments are to be considered in all respects as illustrative and not restrictive. The scope of the invention is, therefore, indicated by the appended claims rather than by the foregoing description. All changes which come within the meaning and range of equivalency of the claims are to be embraced within their scope.

1. An apparatus for controllably releasing a substance, the apparatus comprising:
a chamber to store a non-gaseous fluid;
a outlet in communication with a bottom of the chamber to dispense the non-gaseous fluid therethrough;
an inlet in communication with a top of the chamber to enable gas to flow into the chamber; and
a regulator element coupled to the inlet to regulate the flow of gas through the inlet, thereby regulating the flow of the non-gaseous fluid through the outlet.

2. The apparatus of claim 1, further comprising an emanator to receive the non-gaseous fluid from the outlet, the emanator dispersing the non-gaseous fluid into the atmosphere.

3. The apparatus of claim 2, wherein the emanator comprises a device selected from the group consisting of a wicking device, a heater, a piezo device, a nebulizer, a fan, and combinations thereof.

4. The apparatus of claim 1, wherein the regulator element is a tube having a length and inside diameter selected to provide a desired flow rate through the outlet.

5. The apparatus of claim 4, wherein the tube is formed in one of a spiral, helical, and tortuous shape.

6. The apparatus of claim 4, wherein the tube is at least partially filled with a porous media.

7. The apparatus of claim 1, wherein the regulator element is a gas-permeable membrane.

8. The apparatus of claim 1, wherein the regulator element is a helical path having a length and cross-sectional area selected to provide a desired flow rate through the outlet.

9. The apparatus of claim 1, wherein the regulator element comprises a solid having a channel formed therein.

10. The apparatus of claim 9, wherein the channel is a tortuous channel.

11. The apparatus of claim 1, wherein the chamber includes a short wide portion situated above a relatively long narrow portion, wherein the short wide portion and long narrow portion together generate more consistent head pressure at the outlet.

12. A method for controllably releasing a substance, the method comprising:
storing a non-gaseous fluid in a chamber;
dispensing the non-gaseous fluid though an outlet in communication with a bottom of the chamber;
enabling gas to flow into the chamber through an inlet in communication with a top of the chamber; and
regulating the flow rate of the non-gaseous fluid through the outlet by regulating the flow of gas through the inlet.

13. The method of claim 12, further comprising using an emanator to disperse the non-gaseous fluid into the atmosphere after it has exited the outlet.

14. The method of claim 13, wherein the emanator comprises a device selected from the group consisting of a wicking device, a heater, a piezo device, a nebulizer, and combinations thereof.

15. The method of claim 12, wherein regulating the flow of gas through the inlet comprises passing the gas through a tube having a length and inside diameter selected to provide a desired flow rate.

16. The method of claim 15, wherein the tube is formed in one of a spiral, helical, and tortuous shape.

17. The method of claim 12, wherein regulating the flow of gas through the inlet comprises passing the gas through a gas-permeable membrane.

18. The method of claim 12, wherein regulating the flow of gas through the inlet comprises passing the gas through a helical path having a length and cross-sectional area selected to provide a desired flow rate.

19. The method of claim 12, wherein regulating the flow of gas through the inlet comprises passing the gas through a channel formed in a solid.

20. The method of claim 19, wherein the channel is a tortuous channel.

21. The method of claim 12, wherein the chamber includes a short wide portion situated above a relatively long narrow portion, wherein the short wide portion and long narrow portion together generate more consistent head pressure at the outlet.
